(19) United States
(12) Patent Application Publication
(10) Pub. No.: US 2007/0203426 A1
(43) Pub. Date: Aug. 30, 2007

(54) Method and apparatus for obtaining real time emotional response data over a communications network

(76) Inventors: Arthur J. Kover, Clintin, CT (US); Owen Davis, Flemington, NJ (US); Richard R. Berke, Doylestown, PA (US)

Correspondence Address: Steven S Payne;Suite 1005, 1101 17th Street NW, Washington, DC, 20036, US

(21) Appl. No.: 11/583,985

(22) Filed: Oct. 19, 2006

Related U.S. Application Data
(60) Provisional application No. 60/728,727, filed on Oct. 20, 2005.
(60) Provisional application No. 60/812,005, filed on Jun. 8, 2006.

(51) Int. Cl. A61B 13/00; A61B 5/00

(52) U.S. Cl. 600/558; 600/559

(57) ABSTRACT

A method and apparatus for obtaining real time emotional response data over a communications system is disclosed. A stimulus is presented to at least one participant using the communications system. Emotional response data for each participant is recorded while the stimulus is being displayed. The stimulus can be a visual and/or audio presentation such as an advertisement with static or moving images, marketing information, brochures, sales information, live or recorded speeches, debates, television programs, movies, videos, music, computer graphics, computer games or any other media which can be projected audioly and/or visually over a communications network. The recorded emotional response data is analyzed to determine at least one critical emotion range in the stimulus.

### FIELD OF THE INVENTION

The present invention relates generally to obtaining real time emotional response data of a stimulus such as a presentation. More particularly, the present invention relates to a method and apparatus for obtaining real time emotional response data of a stimulus over time using a communications network such as the Internet to establish Critical Emotion Ranges (CER) in the stimulus.

### BACKGROUND OF THE INVENTION

The objective of marketing research seems rather straightforward, predict how a stimulus will be perceived in the real world. However, it is not really that easy, as the reactions to stimulus are often very complex and not completely understood.

The effectiveness of a stimulus, e.g., an advertisement, a political message, a political speech or debate, can be very hard to determine since it is based on human reactions to the stimulus. The effectiveness of the stimulus will vary from person to person depending on each person's different views and beliefs. Furthermore, the effectiveness of the stimulus may be based on one or several critical ideas or images conveyed during the stimulus. For example, an idea which is described for 10 seconds during a five minute speech may cause such an emotional response (either positive or negative) in a person that the person's entire opinion of the speech will be based on their reaction to the idea described in the 10 second segment. These important emotional responses are called critical emotional responses.

As a result, it is very important not only to determine the opinions of people who have watched or heard the stimulus but also to be able to determine any critical emotional responses that each person experienced while watching or hearing the stimulus. Unfortunately, it can be very hard to adequately identify all of the critical emotional responses after a person has finished watching the stimulus. For example, a person may forget critical emotional responses which occur early in a stimulus or their thoughts and opinions about certain sections of the stimulus may be colored by critical emotional responses which occur at some point during the stimulus.

Accordingly, it is desirable to provide a method and apparatus for obtaining real time critical emotional response data for a stimulus. Furthermore, it is an objective of the invention to measure real time critical emotional response data for a multitude of different types of stimulus which can be audioly and/or visually over a communication network such as the Internet.

### SUMMARY OF THE INVENTION

It is therefore a feature and advantage of the present invention to provide a method and apparatus for obtaining real time critical emotional response data using a communications system such as the Internet. The present invention combines a point-and-click feature and the interactivity of the Internet to capture a persons real-time impressions and thoughts of a stimulus.

In accordance with one embodiment of the present invention, a method and apparatus for obtaining real time critical emotional response data for a stimulus over a communications system is disclosed. A stimulus is presented at least once to a participant. The participant's reactions are recorded while the stimulus is being displayed. The recorded emotional response data is then analyzed to determine at least one critical emotion range in the stimulus. The stimulus can be a visual and/or audio presentation such as an advertisement with static or moving images, marketing information, brochures, sales information, live or recorded speeches, debates, television programs, movies, videos, music, computer graphics, computer games or any other media which can be projected audioly and/or visually over a communication network.

There has thus been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The invention will now be described, by way of example, with reference to the accompanying drawings, wherein:

FIG. 1 illustrates a computer system according to one embodiment of the invention;

FIG. 2 illustrates a screen shot according to one embodiment of the invention;

FIG. 3 illustrates a flow chart showing the operation of the computer system according to one embodiment of the invention;

FIG. 4 illustrates a flow chart for calculating critical emotion ranges according to one embodiment of the invention;

FIG. 5 illustrates a 2-dimensional array of emotional response data for three participants according to one embodiment of the invention;

FIG. 6 illustrates a 2-dimensional array of the averaged emotional response data according to one embodiment of the invention;

FIG. 7 illustrates a 2-dimensional array according to one embodiment of the invention; and

FIG. 8 illustrates a 2-dimensional array according to one embodiment of the invention.

### DETAILED DESCRIPTION OF PREFERRED EMBODIMENTS OF THE INVENTION

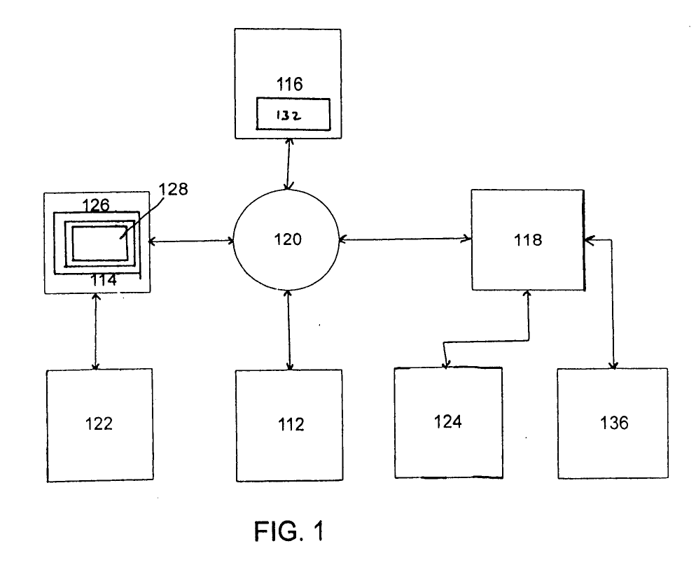

FIG. 1 illustrates an exemplary system 100 for obtaining real time emotional response data for a stimulus over a communications network, such as the Internet, according to one embodiment of the invention. As described more fully below, the system 100 allows a multitude of participants to view a stimulus such as an advertisement with static or moving images, marketing information, brochures, sales information, live or recorded speeches, debates, television programs, movies, videos, music, computer graphics, computer games or any other media which can be projected audioly and/or visually over the Internet and record their real-time reactions and thoughts about the stimulus through a series of requests and questions based on their reactions.

Using the interactivity of the Internet, the present invention can provide precise, effective web-based real time emotional response data on stimulus. The present invention can provide powerful insights into what issues or statements spark the most positive or negative response from a group of participants. The present invention can enrich knowledge, and speed and enhance the decision making process within the framework of qualitative research without the expense and problems associated with other methods.

According to one embodiment of the invention, the research participant is asked to view a stimulus over a network such as the Internet and record their reactions to the stimulus using a mouse or keyboard controls by clicking on a Likert scale positioned, for example, underneath a window containing the video and/or audio stimulus. The system then uses the recorded reactions to calculate where critical emotion ranges occur in the stimulus. The critical emotion ranges are the sections of the stimulus which cause the most extreme responses (both positive and negative) from the participants. The participant can then be asked a series of questions regarding the stimulus based at least partially on the determined critical emotion ranges. It will be understood by those skilled in the art that the stimulus can be a video, slide show, animation, flash animation, or any other type of stimulus that changes over time.

The exemplary system 100 includes a website owner 112, a web server 114, one or more website participants 116, and a reporting server 118 coupled to one another using a network 120. It will be understood by those skilled in the art that the network may be any suitable local area network (LAN), metropolitan area network (MAN), wide area network (WAN), a global communications network such as the Internet, or any other suitable network. Although the owner 112, the server 114, the participants 116, and the server 118 are described as coupled using a single network 120, the present invention contemplates multiple networks 120 of the same type or different types to couple these components together, according to particular needs. The owner 112 and the participants 116 may each be autonomous computer systems or may receive appropriate input from one or more associated persons. The servers 114 and 118 may include software operating on one or more computer systems 122 and 124, respectively, at one or more locations. The owner 112, the server 114 and the server 118 may operate on at least one shared computer system. The computer systems associated with the owner 112, the participants 116, the servers 114 and 118 include input devices, output devices, processors, memories, and other components suitable for the features and operations described below.

The web server 114 hosts or otherwise supports at least one website 126 including one or more pages 128. Although the pages 128 are described primarily as web pages 128 associated with a typical website 126, the present invention contemplates measuring and reporting user reactions to video, animation, flash animation, slide show or any other type of moving stimulus. Moreover, although a single website 126 for a single owner 112 is described in detail, the server 114 may support one or more websites 126 for each of multiple owners 112.

In general, using an associated web browser or other software component, the participant 116 provides a uniform resource locator (URL) or other electronic address to establish a connection to the server 114 and access a particular page 128 associated with the website 126. The server 114 communicates the requested page 128 to the participant 116 using the network 120, the participant 116 receives the page 128, and the participant 116 views or otherwise processes the page 128 according to the participant's particular needs. The participant 116 will typically provide one or more additional URLs during a single browser session to access additional pages 128 associated with the website 126, navigating through the topography of the website 126 according to particular needs. Multiple participants 116 may access a single page 128 substantially simultaneously. The present invention contemplates one or more website participants 116 accessing one or more pages 128 of the website 126 in a suitable manner during one or more browser sessions.

According to one embodiment of the invention, the stimulus is shown to the participant and the participant uses a mouse or various keys on a keyboard or some other input device to select a point on a Likert scale which represents the participant's reactions to the stimulus. The participant's responses are collected, for example in a data array, and then sent via the network 120 to the server 118 and stored in a database 136. Software in the server and/or the associated computer 124 analyzes and interprets the received data as will be described in more detail below.

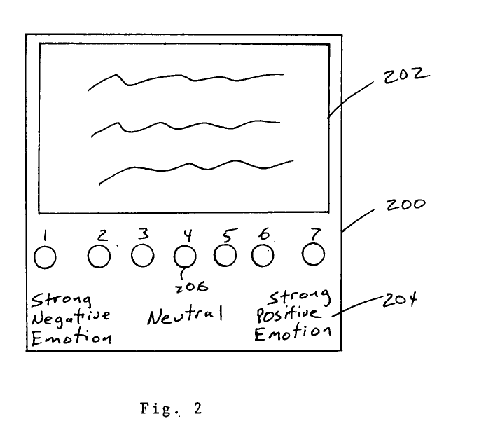

The invention can be employed via the HTTP protocol through a participant's web browser. Flash animations can be embedded within the window providing a Likert scale as well as visual and audio components. For example, the far left side of the Likert scale can indicate the most negative response and the far right side of the Likert scale can indicate the most positive response. As illustrated in FIG. 2, a screen 200 is displayed on the participants computer screen. The screen 200 has an image section or window 202 for displaying the stimulus and a Likert scale 204. The Likert scale 204 has 7 points 206 for indicating the participant's reactions from strongly negative to neutral to strongly positive. While the Likert scale 204 illustrated in FIG. 2 uses 7 points, a Likert scale with any number of points greater than 2 can be used by the invention. Each point on the Likert scale indicates either a generally positive or negative emotion elicited by the stimulus. In this illustrative example, the Likert scale is a horizontal bar below the image section 202. It will be understood by those skilled in the art that the Likert scale can be a horizontal or vertical bar (or some other shape) and can appear anywhere on the screen 200.

The participant can select a point 206 on the Likert scale 204 utilizing his or her mouse as the input device. The participant uses the point and click feature of the mouse to select a numbered response on the scale. The number of clicks and the timing of the clicks are entirely up to the participant. Alternatively, the participant can use keys on a keyboard or any other input device to select a numbered response. For example, the number keys (1-7) on the keyboard could be used to select the numbered response on the Likert scale. According to another embodiment of the invention, the Likert scale 204 can include an indication marker that continuously represents the response of the participant. The participant can manipulate the indication marker utilizes his or her mouse, joystick, keyboard, etc., as the input device. Coincidently, moving the mouse left of its relative position will move the indication marker to the left and vice versa. As a result, the system can continuously record the participant reactions.

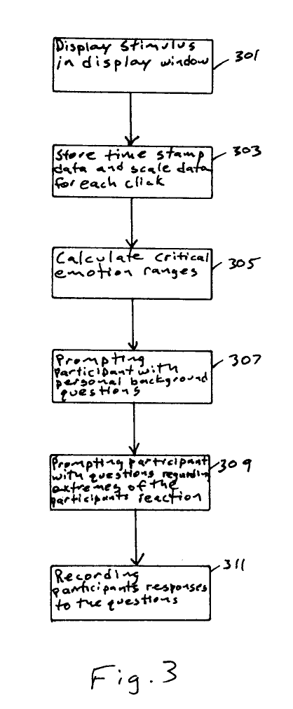

A method for obtaining real time emotional response data over the Internet according to one embodiment of the invention will now be described with reference to FIG. 3. It will be understood by those skilled in the art that any number of steps illustrated in FIG. 3 can be skipped or the order of the steps can be changed without departing from the scope or spirit of the invention. When a participant 116 enters the website 126 and agrees to participate in the research survey, access to a plurality of web pages and other tools are downloaded to the participant's computer 116. One such tool 132 includes data gathering functions that record all of the data entered by the participant 116 during the survey.

In the exemplary embodiment described below, the participant 116 is asked to view and/or listen to a stimulus, for example, a political speech. In step 301, the political speech is displayed in the window 202. As the participant watches and listens to the speech, the participant can click on the scale points 206 at any time during the speech. The number of clicks and the timing of the clicks are entirely up to the participant. As the participant clicks the scale points 206, time stamp data and scale data are stored by the system 100 in step 303. In this embodiment of the invention, data is only collected when a click occurs, but the invention is not limited thereto. For example, the system can also continuously record the participant's reactions as the participant manipulates the indication marker. According to another embodiment of the invention, the velocity of the movement of the mouse can be used to determine rapid changes in the participant's emotions.

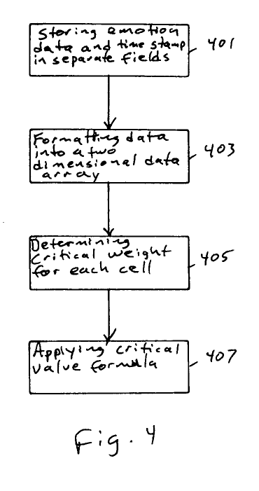

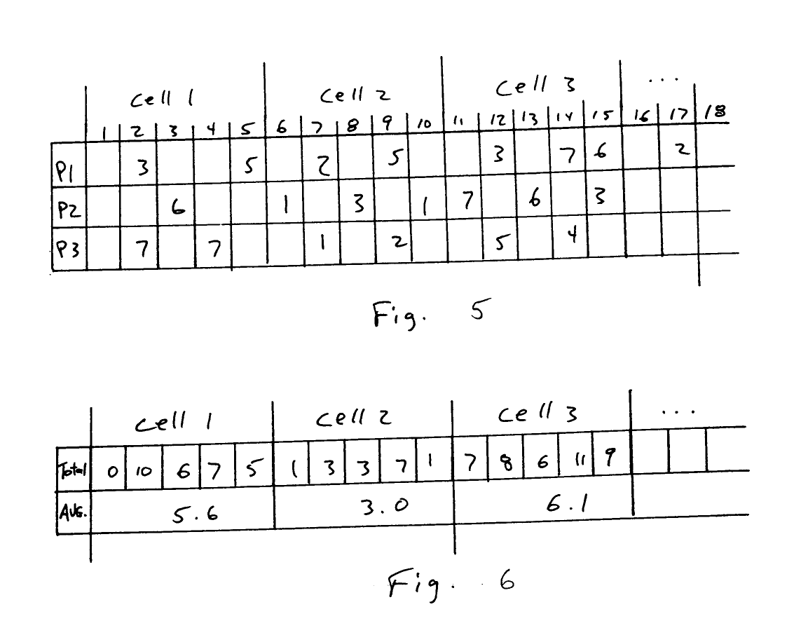

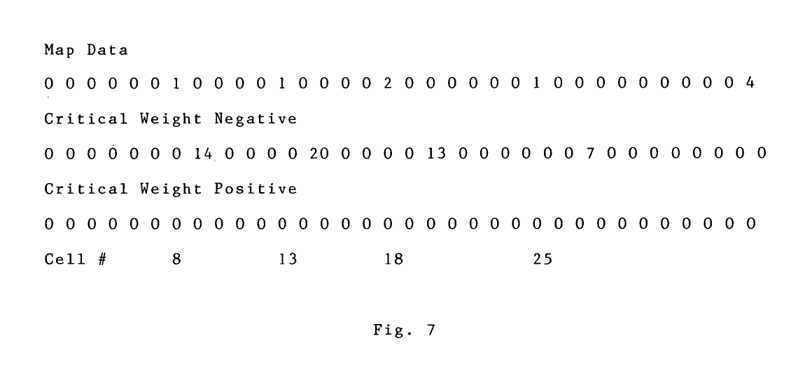

Once the speech is over, all of the data from all of the participants is gathered and critical emotion ranges for the speech are calculated in step 305. FIG. 4 illustrates one method for calculating the critical emotion ranges according to one embodiment of the invention. In step 401, emotion data is stored numerically from 1-7 in a single field and time stamps are stored concurrently in a separate field and represent the frame of the stimulus. The frames per second of the stimulus can vary by stimulus. The data is then formatted into a two dimensional data array that will compress and average the data in ColumnLength sections, in step 403, wherein ColumnLength is defined as the cell length in seconds. In other words, the speech is divided into a series of cells, each cell being X seconds in length. FIG. 5 illustrates how the emotion response data for three participants is placed in a two dimensional array and divided into a plurality of cells. The emotion data for all participants in each cell are added together and averaged as illustrated in FIG. 6.

In step 405, the critical weight for each cell is evaluated. Data is transformed into a new data array where each cell represents the averages of all values+/−CriticalDistance cells where the cell value is not neutral. Only cells that contain critical values above and below two threshold values are kept, for example, cells that contain critical values in the bottom 30% and the top 70% of the Likert scale. Critical weights are indicators of emotional hot spots. Alternatively, the critical weight for a cell can be determined by summing together weight values within a predetermined range of the cell in question. As illustrated in FIG. 7, the critical weight for each cell is calculated by adding together weight values within 5 cells, both forward and previous of the cell in question. In this example, a cell which has received a strongly negative rating of a 1 is assigned a negative weight of 7 and a rating of 2 is assigned a negative weight of a 6. Likewise, a cell which has received a strongly positive rating of a 7 is assigned a positive weight of 7 and a rating of 6 is assigned a positive weight of a 6. In this example, cells which receive ratings of 3-5 are ignored. The critical weight for cells with ratings of 1 or 2 is then determined by adding the weight values of the cells within 5 cells each side of the cell in question and including the cell in question. For example, cell 8 has a critical negative weight of 14, 7 for cell 8 and 7 for cell 13. Likewise, cell 13 has a critical negative weight of 20, 7 for cell 8, 7 for cell 13 and 6 for cell 18. The rest of the critical weights are calculated in the same manner.

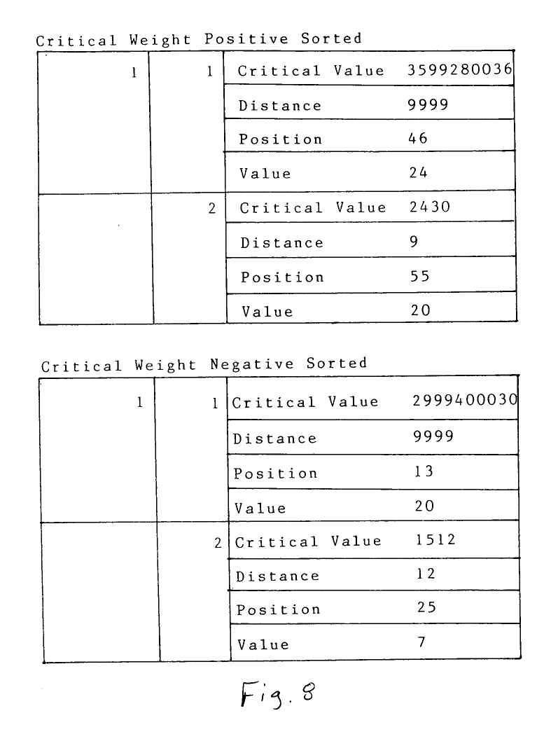

A critical value formula is then applied in step 407 to all items in the critical weight array where CV (critical Value)=(Critical Weight*((Distance from cell N)²*Distance Weight)) and sorted from highest to lowest in a final 2 dimensional array. Positive and negative emotions are split and stored in separate arrays as illustrated in FIG. 8. Distance restrictions can be enforced so that not all cells which are grouped closely together are used as Critical values.

Returning to FIG. 3, once the critical emotion ranges have been determined, each participant can be prompted to answer a series of questions over the communications system regarding their personal background and question related to the stimulus and the critical emotion ranges in step 307. For example, each participant can be asked to enter their age, sex, race, religion, income, political affiliations, marriage status, how likely they are to vote or vote for a particular candidate, etc. These questions about the participant's personal background can be tailored depending on the subject matter of the stimulus being displayed. Specific follow-up questions about the stimulus can also be displayed. For example, questions can be asked about specific sections of the stimulus which were determined to be critical emotion ranges. In this instance, the sections of the stimulus which correspond to at least one of the determined critical emotion ranges can be shown again to the participant and the participant can be asked why this section or sections of the stimulus invoked such extremes in the participant's reactions in step 309. The system 100 records the participant's responses to the questions in step 311. The responses can then be analyzed in a multitude of ways to gain valuable statistical information about the effectiveness of the stimulus in conveying different types of messages.

As mentioned above, the system 100 can be used to collect real time data on a wide variety of presentations such as an advertisement with static or moving images, marketing information, brochures, sales information, live or recorded speeches, television programs, movies, news programs or segments, videos, music, auditions computer graphics, computer games or any other media which can be projected audioly and/or visually over a communication system. In addition, research data on audio only presentations can also be obtained by playing the audio presentation over the network 120 while the participant records their reactions using the Likert scale 204 as described above.

The many features and advantages of the invention are apparent from the detailed specification, and thus, it is intended by the appended claims to cover all such features and advantages of the invention which fall within the true spirits and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method for obtaining real time emotional response data for a stimulus over a communications system, comprising the steps of:
presenting the stimulus to at least once to a participant using the communications system;
recording emotional response data for each participant while the stimulus is being presented;
analyzing the recorded emotional response data to determine at least one critical emotion range in the stimulus.

2. The method according to claim 1, wherein the stimulus is from the group comprising an advertisement with static or moving images, marketing information, brochures, sales information, live or recorded speeches, debates, television programs, movies, news programs or segments, videos, music, auditions, computer graphics, computer games.

3. The method according to claim 1, further comprising the step of:
posing at least one question to said at least one participant regarding at least one critical emotion range.

4. The method according to claim 3, wherein a section of the stimulus which corresponds to the at least one critical emotion range is re-presented to the participant using the communications system.

5. The method according to claim 3, further comprising the step of:
posing at least one question regarding the participant's personal background.

6. The method according to claim 5, further comprising the steps of:
analyzing responses to said questions;
producing a statistical analysis of the stimulus based on said responses to said questions and said critical emotion ranges.

7. The method according to claim 1, wherein said emotional response data comprises a numerical value which represents the participants' emotion and a time stamp value.

8. The method according to claim 7, wherein the numerical value ranges from 1 to 7.

9. The method according to claim 7, wherein said at least one critical emotion range is determined by the following steps:
dividing the stimulus into a plurality of cells;
calculating an averaged response value for emotional response data of all participants that occurred in each cell;
discarding cells which have averaged values between first and second threshold values, wherein remaining cells indicate critical emotion ranges.

10. The method according to claim 9, further comprising the step of:
discarding cells which occur within a predetermined distance from a cell which indicates a critical emotion range.

11. An apparatus for obtaining real time emotional response data for a stimulus over a communications system, comprising:
means for presenting the stimulus to at least once to a participant using the communications system;
means for recording emotional response data for each participant while the stimulus is being presented;
means for analyzing the recorded emotional response data to determine at least one critical emotion range in the stimulus.

12. The apparatus according to claim 11, wherein the stimulus is from the group comprising an advertisement with static or moving images, marketing information, brochures, sales information, live or recorded speeches, debates, television programs, movies, news programs or segments, videos, music, auditions, computer graphics, computer games.

13. The apparatus according to claim 11, further comprising:
means for posing at least one question to said at least one participant regarding at least one critical emotion range.

14. The apparatus according to claim 13, wherein a section of the stimulus which corresponds to the at least one critical emotion range is re-presented to the participant using the communications system.

15. The apparatus according to claim 13, further comprising:
means for posing at least one question regarding the participant's personal background.

16. The apparatus according to claim 15, further comprising:
means for analyzing responses to said questions;
means for producing a statistical analysis of the stimulus based on said responses to said questions and said critical emotion ranges.

17. The apparatus according to claim 11, wherein said emotional response data comprises a numerical value which represents the participants' emotion and a time stamp value.

18. The apparatus according to claim 17, wherein the numerical value ranges from 1 to 7.

19. The apparatus according to claim 17, further comprising:
means for dividing the stimulus into a plurality of cells;
means for calculating an averaged response value for emotional response data of all participants that occurred in each cell;
means for discarding cells which have averaged values between first and second threshold values, wherein remaining cells indicate critical emotion ranges.

20. The apparatus according to claim 19, further comprising:
means for discarding cells which occur within a predetermined distance from a cell which indicates a critical emotion range.
